(19) United States
(12) Patent Application Publication
(10) Pub. No.: US 2005/0043750 A1
(43) Pub. Date: Feb. 24, 2005

(54) Laparoscopic stone safety device and method

(76) Inventors: George L. Scott III, Cedar Crest, NM (US); James C. Rosser, Rye Brook, NY (US); Donald E. Wenner, Roswell, NM (US)

Correspondence Address: Loren G. Helmreich;Browning Bushman, P.C., Suite 1800, 5718 Westheimer, Houston, TX, 77057, US

(21) Appl. No.: 10/866,588

(22) Filed: Jun. 11, 2004

Related U.S. Application Data
(60) Provisional application No. 60/477,871, filed on Jun. 13, 2003.

(51) Int. Cl. A61B 17/08

(52) U.S. Cl. 606/151

(57) ABSTRACT

A laparoscopic netting assembly is provided for conducting a gallbladder or bile duct procedure through one of the throughbores in a carrier sheath, which in turn may be positioned within a laparoscopic port. The carrier sheath includes at least one through channel for conducting a frame control rod, and optionally a deployment rod. The netting assembly includes a collapsible and expandable frame and a fluid permeable netting, which is preferably comprises a plurality of netting layers, suspended on the frame for collecting stones released from the gallbladder or bile duct. The frame is sized to also collect the gallbladder. The carrier sheath may also include one or more through channels for a cutting instrument.

### FIELD OF THE INVENTION

The present invention generally relates to medical equipment and, more particularly, to a laparoscopic surgical instrument of the type used in gallbladder and biliary tract exploration and stone extraction procedures. This surgical safety device may reliably prevent migration of stones from the gallbladder, or the bile duct hepatobiliary tree and the common bile duct.

### BACKGROUND OF THE INVENTION

Many patients develop stones within their gallbladder. A diseased gallbladder may contain dozens or several hundred stones and typically is removed by open surgery or minimally invasive laparoscopic cholecystectomy. The gallbladder is often cut or torn during the laparoscopic cholecystectomy procedure. As a result, stones may be spilled into the peritoneal cavity of the abdomen. This undesirable event may occur in approximately 10% to 40% of procedures performed. Without the invention, unrecovered stones adjacent to the abdomen, liver, or other vital organs may thus be closed within the patent at the end of the surgical operation, and the subsequent location and removal of those stones is at best difficult and expensive. Unrecovered stones in the retrohepatic region may, for example, create delayed complications for the patient, including abscess or fistula formation which is hazardous to the patient's health and recovery. Multiple stones may be displaced from the gallbladder or bile duct, and may subsequently migrate into the space behind the liver, which results in more of surgeon's time spent trying, often unsuccessfully, to retrieve these extra biliary stones.

Spilled stones typically migrate to a location that is generally bordered by the common bile duct and portal vein, laterally by the chest wall and diaphragm, superiorly by the liver, inferiorly by the hepatic flecture of the colon and the C-loop of the duodenum, and posteriorly by the retroperitoneum and right kidney. A surgeon typically removes the loose stones using suction, grasping or scooping instruments. Often these stones remain lost, even after numerous diligent attempts by the surgeon. Unrecoverable stones may become a source of infection, and have been reported to fistulize through the diaphragm and even through the skin in the flank region. Lost stones may thus be a source of significant morbidity and potential liability to the surgeon. During laparoscopic common bile duct exploration (LCBDE), stones removed from the bile duct may inadvertently fall into a same space behind the liver and above the kidney. Stones may be broken with the lithotripter or laser, and are often fragmented. During laparoscopic cholecystectomy procedures, unrecovered whole stones and fragmented stones tend to migrate to the area beneath the liver, and on the right side of the abdomen, where subsequent laparoscopic retrieval is very difficult. Conversion from a laparoscopic procedure to open surgery may be necessary when stones cannot be retrieved.

The process of locating and removing misplaced stones or associated stony debris is often difficult, since visualization and exposure via laparoscopic techniques is inadequate. The search process may be frustrating, tedious and stressful to a surgeon. Additional manipulation of the patient's liver may be required during the errant stone exploration process to find small stones that fall from the gallbladder or bile duct, which may result in additional organ trauma, including bleeding from the liver. In patients with extensive tissue adhesions, the search often requires extensive operating time.

Prior art procedures for removal of gallstones and stones located in the biliary tree present the practitioner with an increasingly complex and time-consuming problem when stones are lost. Too frequently, a laparoscopic surgeon may fail to achieve the desired goal of complete stone removal even after extensive operational time. A medical retrieval device with a basket formed from two or more loops is disclosed in U.S. Pat. No. 6,520,968. An article relevant to this invention is entitled “Jaundice Due to Extrabiliary Gallstones”, Stevens, et al., Vol. 7, Number 3, JSLS, 277 (July-September 2003).

The disadvantages of the prior art are overcome by the present invention, and an improved laparoscopic stone safety device and method are hereinafter disclosed which should significantly reduce the hazardous risk of unrecovered stones.

### SUMMARY OF THE INVENTION

The present invention may be used when performing laparoscopic procedures related to exploration and the removal of physiologic calculi (“stones”) from the hepatobiliary tract, including the gallbladder and the common bile duct. The laparoscopic surgical tool safely collects stones loosed from the gallbladder and biliary tract that otherwise would be inadvertently spilled into the patient, thereby preventing complications that otherwise may occur when stones migrate into the free peritoneal space of the abdomen. This surgical safety instrument thus blocks migration of stones and protects the patient from morbidity and trauma to organs, including the liver, by avoidance of excessive manipulation that is otherwise involved when the surgeon searches for lost stones. The safety device also minimizes operative time, since searching for lost stones is eliminated or minimized.

It is a feature of preferred embodiments to provide the practitioner with a versatile laparoscopic surgical safety instrument to enhance the surgeon's success rate at recovering stones and thus lowering patient trauma and risk.

It is also a feature to provide an improved laparoscopic surgical netting assembly for conducting a laparoscopic gallbladder or bile duct procedure, which may be conventionally conducted through a laparoscopic port having an external end extending axially above an external surface of the abdomen wall, and an abdominal end extending from below an internal surface of the abdominal wall and into the abdominal cavity. The laparoscopic port includes an internal throughbore extending between the external end and the abdominal end which provides a conduit into the abdominal cavity. A carrier sheath is received in the laparoscopic port internal through bore, and has a carrier sheath external end and an instrument guide abdominal end. The carrier sheath external end extends above the external surface of the abdominal wall, and a carrier sheath abdominal end extends below the laproscopic port abdominal end and proximal to the gallbladder or bile duct. The carrier sheath includes at least one through channel for conveying and deploying the surgical netting assembly. The surgical netting assembly is comprised of a collapsible and expandable frame that may be compressed and pre-packaged in a tubular deployment sheath. The frame may be expanded by extension from deployment sheath to define a perimeter substantially greater than the collapsed frame. In one embodiment, a fluid permeable netting suspended on the frame collects stones released from the gallbladder or bile duct, while allowing fluid to passthrough the netting during retrieval of the netting assembly. The method of the invention will be apparent from the disclosure of a preferred embodiment.

It is a feature that the frame may be formed with a memory that defines substantially the expanded frame. The expanded frame may be fabricated to exhibit memory curvature and may have an oval configuration with saddle-shaped geometry observable in side-view. In a preferred embodiment, the long axis of the oval configuration is substantially parallel to a central axis of the instrument guide, while the short axis of the oval is perpendicular to the central axis of the instrument guide. The long axis may be from about 3″ to 5″, and the short axis from 1″ to 3″. A top surface of the netting may be provided ½″ or more below the short axis at its midpoint. The netting assembly's depth and frame geometry may be modified selectively by the surgeon as a function of the amount of extension from the deployment sheath.

A further feature is that the netting may comprise two or more netting layers spaced apart when the frame is in its deployed position. A lower netting layer has a smaller passthrough area than an upper netting layer area. In a preferred embodiment, three or more layers are provided, with each layer spaced from an adjacent layer when the frame is in its deployed configuration. In one embodiment, the netting may comprise loop strands with individual loops substantially perpendicular to the frame of the netting. The frame may be returned to substantially its collapsed position during retrieval. In one embodiment, an elongate tether is secured to the frame for assisting in the retrieval of the netting assembly.

It is a feature that the netting assembly may have a frame consisting of an outer frame member and an inner frame member spaced within the outer frame member. The outer frame member may include an elongate outer wire, with both ends of the outer wire passing through the at least one through channel in the carrier sheath, such that the elongate outer wire may be extended and retracted within the at least one through channel in the carrier sheath. The inner frame member may include an inner frame wire, with both ends of the inner frame wire similarly passing through the at least one through channel in the carrier sheath. The outer frame wire and the inner frame wire are separately extendable and retractable within the at least one through channel for changing the configuration of the frame. The outer frame may support a fine mesh netting, and the inner frame may support a course mesh netting positioned above the fine mesh netting.

In another embodiment, the netting assembly is provided with a fluid permeable netting suspended on the frame. The frame and netting are sized for collecting the gallbladder and one or more stones released from the gallbladder or bile duct. The frame may also support a fluid impermeable layer for collecting fluid from the gallbladder or bile duct.

In yet another embodiment, the carrier sheath is provided with a plurality of through channels, with one of the channels receiving the frame and netting. A surgical tool passes through another of the plurality of channels in the carrier sheath, with a surgical tool comprising one of a scalpel, scissors, or cutting device.

According to a method of the invention, both the gallbladder and one or more stones released from the gallbladder or bile duct may be collected in the netting of the frame.

These and further objects, features and advantages of the present invention will become apparent from the following detailed description, wherein reference is made to the figures in the accompanying drawings.

### BRIEF DESCRIPTION OF THE DRAWINGS

FIG. 1 is a pictorial view, partially in cross-section, of a surgical netting assembly for deployment from a carrier sheath in the abdomen.

FIG. 2 is a pictorial view of the fully deployed netting assembly shown in FIG. 1 with a single netting layer.

FIG. 3 illustrates a netting assembly partially deployed, and FIG. 4 illustrates the same netting assembly further but yet not fully deployed.

FIG. 5 illustrates a threaded connector between the netting assembly s deployment rod and frame

FIG. 6 illustrates a permeable foam membrane netting layer.

FIG. 7 illustrates another embodiment of a permeable netting layer.

FIG. 8 is a cross-sectional view of an outer membrane netting layer and multiple fiber layers for a central netting layer.

FIG. 9 illustrates a netting assembly with frame partially deployed via a basket deployment rod.

FIG. 10 illustrates the netting assembly as shown in FIG. 9 further deployed by movement of the outer deployment rod relative to the inner configuration control rod.

FIG. 11 illustrates in cross-sectional view of progressively smaller passthrough area netting layers for a netting assembly.

FIG. 12 illustrates in cross-section an alternative netting assembly with looped strands.

FIG. 13 illustrates a cross-sectional view of another embodiment of the assembly of the present invention.

FIG. 14 is a cross-sectional view to the carrier sheath shown in FIG. 13.

### DETAILED DESCRIPTION OF PREFERRED EMBODIMENTS

The inventive device provides a membrane that acts as a trapping mechanism to immobilize, block or trap stones and stone fragments as they emerge from the gallbladder and bile ducts, thus preventing undesirable migration into the abdominal cavity. The membrane, which may be a thin layer, or of various three dimensional geometric configurations, or combination thereof, covers the subhepatic space in the abdomen to prevent the stones from migrating after emergence from either the gallbladder or biliary tract. This invention blocks stone migration and thus reduces patient trauma and the common complications that are associated with stone retrieval from laparoscopic cholecystectomy and laparoscopic common bile duct exploration procedures.

The netting assembly may be compressed and pre-packaged in a carrier tube sheath that is deployed by insertion through a carrier sheath within a laparoscopic port. The distal end of the device may extracted from the carrier tube sheath and expanded manually by the surgeon, or by preformed memory, to form a barrier which prevents stone migration beneath the liver. As a result of application of this device, any stones that are displaced from the gallbladder or common bile duct remain blocked from migrating behind the liver. The device with stones trapped therein is removed at the completion of the procedure either by retraction into the carrier sheath or by placement into a specimen bag. This stone-immobilization device thus facilitates the surgeon's ability to remove stones and associated debris with an improved rate of operational success and with a reduced risk of post-operative infection or organ trauma.

The laparoscopic/surgical netting assembly 10 makes use of common laparoscopic port sizes, typically between 5 mm and 12 mm. It will be apparent to those skilled in the art that the configuration and relative positions of deployment of the device is variable and may be tailored to procedural needs and specific anatomical features. Deployment of the device is typically under the gallbladder and to the right side of the common bile duct so as to trap stones and stone fragments, thus avoiding hazardous migration of stones and stone debris during the surgical procedure.

An oval, rounded, or rectangular geometry for the frame may be used with a saddle-shaped configuration, although other geometric configurations, such as polygonal or trapezoidal configuration, may be chosen. In a preferred embodiment, frame 20 when deployed has a generally oval configuration, with a long axis 22, which is substantially parallel to a central axis of the inward end of the instrument guide. The short axis is substantially perpendicular to the long axis. In a preferred embodiment, the long axis is from about 3″ to about 5″ long, and the short axis is from about 1″ to about 3″ wide. The long axis is preferably about 4″ and the short axis of about 2″ is preferred. The uppermost layer of the netting material is preferably at least ½″ or more below the short axis at its midpoint. The substantial size of the frame 21 when expanded is sufficient, in a preferred embodiment, such that the nettings supported on the frame may collect both the gallbladder and one or more stones released from the gallbladder or bile duct. This substantial size also allows a plurality of stones to be easily collected within the netting, which may cover a relatively large area for capturing stones which otherwise may drop into body cavities.

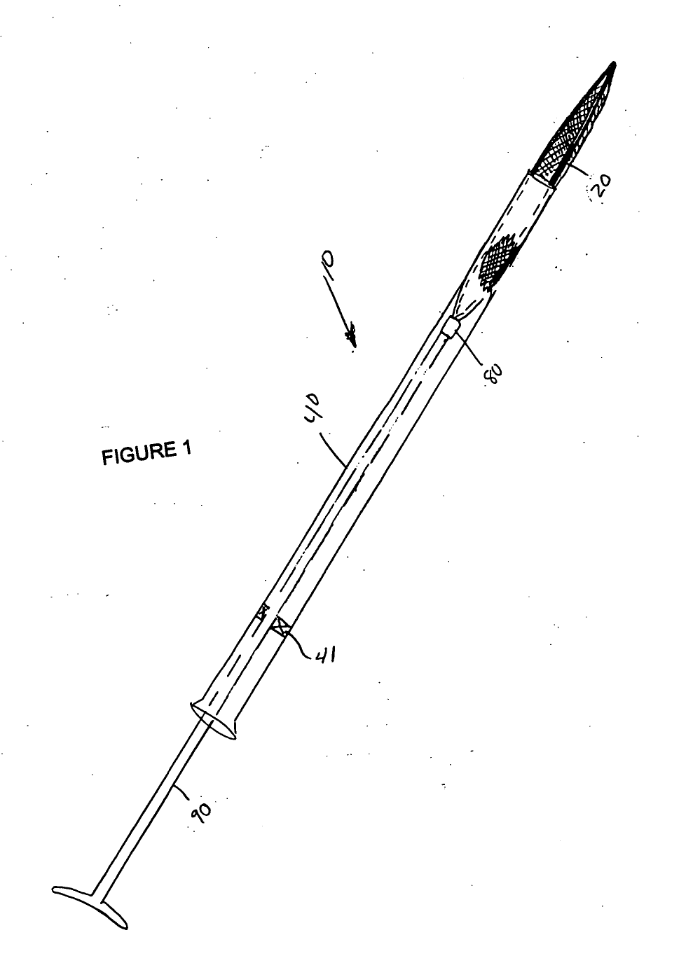

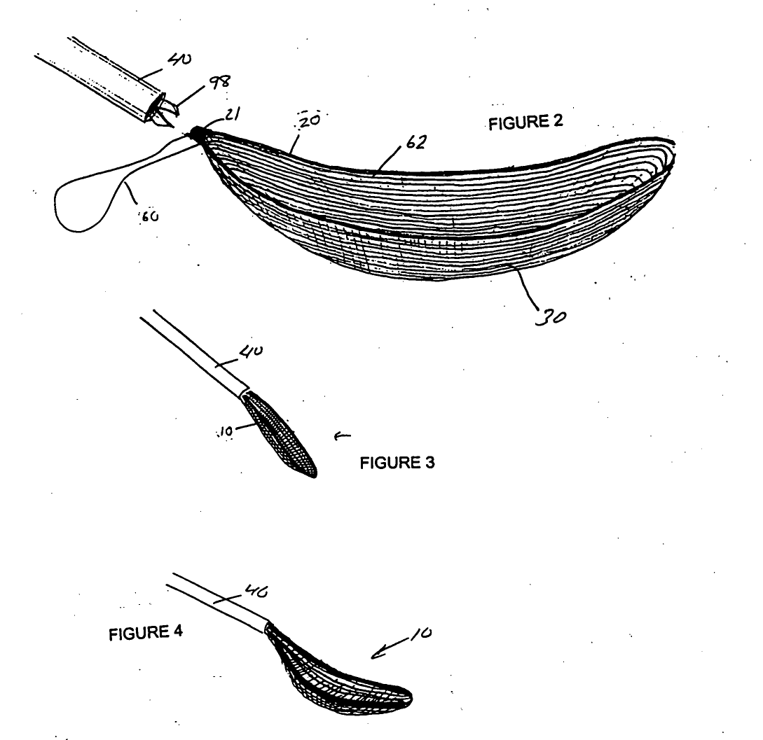

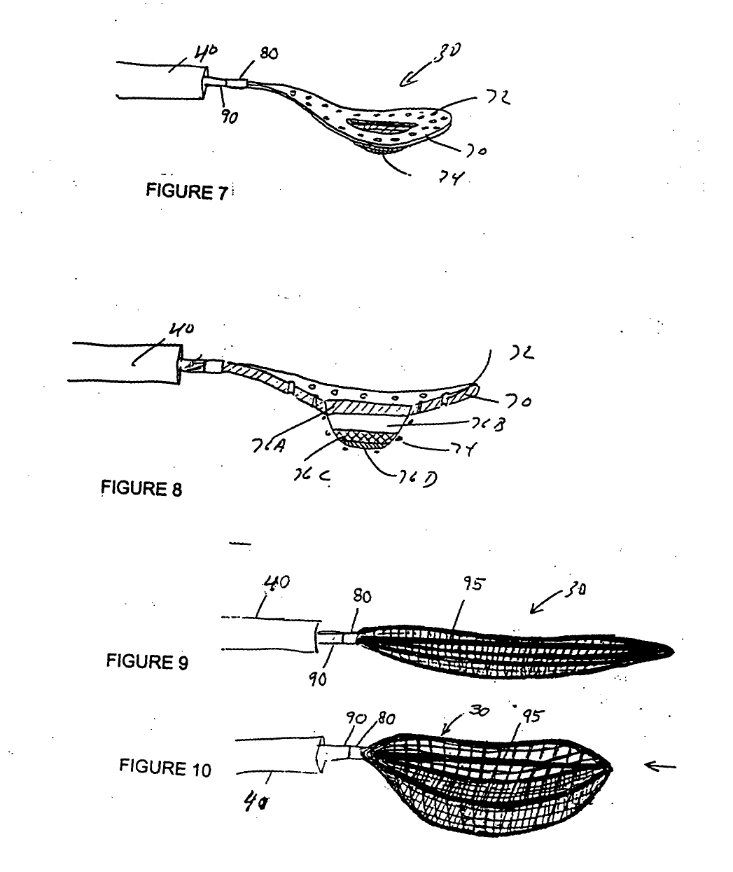

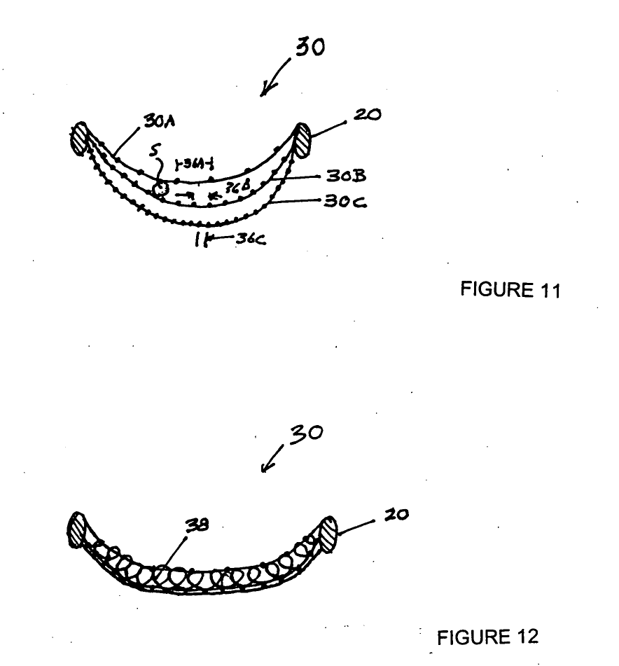

The netting layer 30 as shown in FIG. 2 may include a thin, flat sponge, a pierced membrane, a screen with looped elements, or a netting or mesh material. A netting material is preferred, with the netting strings defining a passthrough area to facilitate passage of fluids, and may include a rectangular, hexagonal, octagonal or other selected configuration. In a preferred embodiment, the netting layer 30 comprises at least two layers and preferably at least three layers with each layer being spaced from an adjacent layer and having a smaller passthrough area, such that stones that effectively become trapped between layers. FIGS. 6 and 7 illustrate a netting assembly and three layers 30A, 30B and 30C each having a rectangular area. The passthrough area is intended for catching the large stones, which typically are about two centimeters in diameter, while passing through the netting small stones which can conventionally be recovered by vacuum, which are typically about 3 millimeters in diameter or less. In an exemplary embodiment as shown in FIG. 11, the netting assembly comprises a frame 20 as discussed above and three netting layers 30A, 30B, and 30C. For this exemplary embodiment, the passthrough areas in the upper layer may have a generally square configuration, so that the minimum diameter stone that may pass through the upper layer may have a diameter approximating 36A as shown in FIG. 1. The second or intermediate layer may have a smaller passthrough area for a minimum diameter of 36B, and the lowest layer 30C may have netting passthrough area of diameter 36C. The passthrough area for the lowest layer may thus be from about 1 millimeters to about 5 millimeter, thereby effectively capturing the smallest of the stones which cannot be conventionally recovered by vacuum. The intermediate layer may have a passthrough diameter 36 B of from about 5 millimeters to 10 millimeters, while the uppermost layer 38 may have a passthrough area of about 10 millimeters to about 2 centimeter.

In the alternate embodiment as shown in FIG. 12, the netting material may form loop strands 38 which extend substantially upward in a direction generally perpendicular to the plane of the netting layer, as shown in FIG. 12. Alternatively, 3-D shapes with concavity or more complex molded configuration for the netting layers may be utilized.

The stone barrier or netting layer 30 and the frame 20 may be compressed or furled in a carrier sheath 40 for insertion through a laparoscopic port. The netting assembly 10 may then be extracted by surgeon from the sheath and opened within the peritoneal cavity. Alternatively, the device may be deployed mechanically via pushing a linear rod 90 down the sheath. Upon deployment into the peritoneum, the device 10 may assume a predetermined shape related to the predetermined memory of the frame. A self-sealing valve 41 as shown in FIG. 1 may be provided to seal between the interior of sheath 40 and the exterior of rod 90, and also to close off flow through the sheath 40 when the rod 90 is removed from the sheath, to prevent escape of gas. The valve 41 may be employed in all the embodiments, but is only shown in FIG. 1. The device may be cinched closed thus trapping stones by pulling a purse string 60 (see FIG. 2) around the perimeter of the stone barrier. Alternative closure methods are envisioned such as twisting, rolling, furling or winding of the stone barrier or attached wire or string. The device 10 with the trapped stones may then be removed from the peritoneal cavity, and optionally may be retrieved through a laparoscopic port.

The permeable membrane 30 may have a high pile, or looped fabric configuration to entrap stone material. One embodiment of the device provides a porous barrier that allows liquid and blood to easily penetrate through, but screens out the stone material. The permeable membrane is preferably formed from a chemical composition that is non-adherent to body tissues.

After insertion of the stone barrier through the port, the surgeon may position the instrument in the space below the gallbladder, to the right of the common bile duct, inferior to the liver, and superior to the hepatic flecture of the colon, thus blocking stone migration to the deep recesses behind the liver. The stone barrier is deployed prior to dislodgement of stones from the gallbladder or bile ducts. The stone barrier is then gathered and removed after the danger of stone spillage into this space has passed.

By minimizing prolonged fishing expeditions for stones spilled into the peritoneal cavity the efficiency of laparoscopic cholecystectomy and laparoscopic common bile duct exploration and the stone removal process may be enhanced and complications related to retained stones within the peritoneal cavity may be eliminated, thus benefitting the patient. The surgical netting assembly collects stones that are inadvertently spilled from the hepatobiliary tract, including the gallbladder and the common bile duct. Alternatively, a suction catheter or stone basket may be introduced through the instrument guide to remove stone debris.

Use of the laparoscopic surgical netting assembly for conducting a laparoscopic gallbladder or bile duct procedure should be apparent to those skilled in the art in view of the above disclosure. The procedure may conventionally be conducted through a laparoscopic port having an external end above the abdominal wall and an internal end within the abdominal cavity, with the laparoscopic port including a throughbore extending between the ends to provide a conduit into the abdominal cavity, and to facilitate introduction of a pneumoperitoneum to insuflasuffrate the abdominal cavity.

The surgical netting assembly may thus be introduced laparoscopically with the frame collapsed and supporting the fluid permeable membrane along its perimeter. Insertion through the carrier sheath 40 is accomplished by pushing, pulling, or rotating the frame control rod 90, which is attached to the netting assembly frame 30 by removable connector 80. The frame 20 may be expanded upon extraction from its carrier sheath 40, whereby its memory assumes a substantially greater area than the collapsed frame. The netting 10 supported on the expanded frame will thus have a sufficient area so that the surgeon may place the netting assembly in its expanded position below the gallbladder and/or bile duct for collecting stones which may then be easily collected and removed. The netting assembly may remain attached to its frame control rod 90 or may be detached via connector 80 to allow the laparoscopic port to be used for other instrumentation. Alternatively, the surgeon may elect to use the netting assembly with a smaller portion of the netting surface area exposed via partial emergence from the carrier sheath, which also allows the device to be used manually by manipulation for scooping stones or stone debris. The geometric configuration of the netting surface may also be controlled and modified by turning and pushing or pulling rod 95. Rod 95, which may also be considered a deployment rod, acts on the frame when expanded, as shown in FIG. 10. The deployment rod is movable with respect to control rod 90 within the same through channel that receives the control rod 90, or the deployment rod 95 may be provided in another one of the through channels within the carrier sheath. The rod 95 engages the frame at a location spaced from the connection of the frame to rod 90, and acts to change the configuration of the frame.

The lateral amount of extension of the netting assembly from the carrier sheath is varied by extension of the frame control rod 90 by the surgeon to selectively control the frame expansion and netting geometry of the device. The netting allows fluids to passthrough the netting during retrieval of the netting assembly and trapped stones. Very small stones that passthrough the netting may be collected by vacuum line.

FIG. 2 shows the generally saddle-shaped oval configuration for a preferred frame 20, wherein the rearward end of the frame preferably has a grasping stud 21 secured to the frame. The end of grasping and releasing tool 98 may thus be used to move the netting assembly through the elongated tube 40 and, as shown FIG. 2, may be activated by the surgeon during release of the netting assembly from the tube 40. If desired, the langard 60 may be connected with the frame, and optionally may by used to assist in retrieval of the netting assembly with the stones captured therein. Also, langard 60 may include a retrieval loop as shown in FIG. 2, which is continuous with a framed loop 62, such that the combination of the retrieval and the frame loops form a continuous looped band. The desirability of this option is that the surgeon may grasp retrieval loop 60 and pull on the retrieval loop, thereby “tightening up” the maximum diameter of the frame loop 62 to effectively cause the frame to collapse about the netting assembly with the captured stones within the netting assembly, in a manner similar to a drawn string on a bag or purse. This feature thus further reduces the likelihood of a stone inadvertently being released from the netting assembly prior to being retrieved from the patient. In another embodiment, the frame may have a generally circular shape, with the netting layer or layers having a generally funnel shaped configuration.

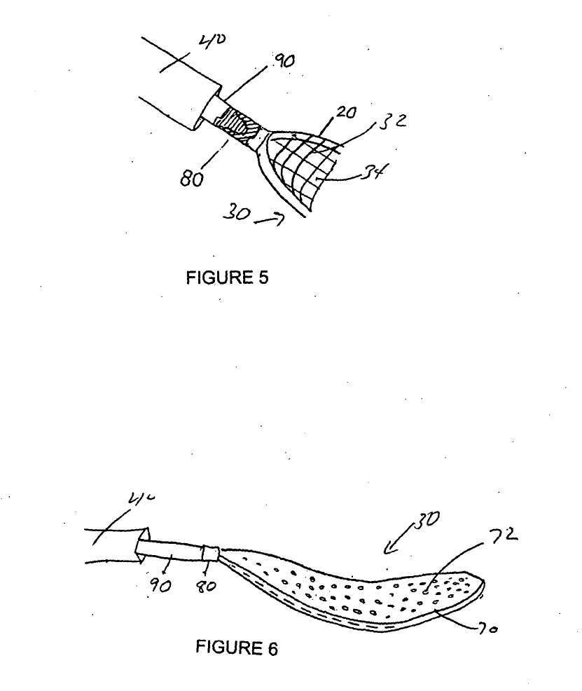

FIG. 5 shows an alternatively threaded connection 80 between the frame control rod 90 and the frame 20. The netting strands 32 may have generally rectangular or squared-shaped passthrough openings 34 as shown.

FIG. 6 disposes an alternative netting assembly, wherein the netting assembly is not a conventional net, but is a net in a sense that it provides a flexible barrier to capture the stones, but it is sufficiently porous to allow blood and other fluids to pass through the netting assembly. Also, the netting assembly 30 as shown on FIG. 6 does not have a frame. The netting assembly 30 is formed from a generally plastic sheet 70 which may be rolled into a small diameter to pass through the tube 40, then unrolled to occupy the substantially larger area for desirably capturing the stones. The plastic layer 70 may have selectively sized passthrough holes 72 for fluid flow, and most of these passthrough holes preferably are generally circular in cross-section to reduce manufacturing cost and to reduce the likelihood of a tear in the sheet 70 during use of the netting assembly.

FIG. 7 discloses yet a further alternative, in which the plastic sheet 70 includes a passthrough center hole 72 with a netting assembly 74 secured to the edge of the large diameter hole. In this embodiment, the netting assembly 74 may catch most of the stones, since the stones will move by gravity toward the net 74 may catch most of the stones, since the stones will move by gravity toward the net 74 due to the contour of the sheet 70.

FIG. 8 discloses yet another embodiment, and again depicts in cross-sections sheet 70 with passthrough holes 72. In this embodiment, the large hole in the center of the plastic sheet 70 is filled with a filtering material, which in one embodiment may be held in place by netting 74. The top layer 76A of the filter is designed to pass the majority of the stones through the layer, so that stones engage the second void layer 76B. A third layer 76C has a still smaller passthrough area, so that most stones will be captured on top of a layer 76C. The last layer 76D has the smallest passthrough area, which is designed to capture the smallest of the stones to be retrieved with the netting assembly. Each of the layers 76A, 76C and 76D may be formed from a fibrous material or a form material, and has the preferable desired flexibility and low cost to achieve the objectives of the invention, while also selectively capturing most stones between different layers, thereby insuring likelihood that the stones will be removed from the patient at the completion of the surgery. Moreover, the various layers 76A, 76C and 76D may be colored coded, if desired, so that a certain color corresponds to a certain passthrough area. If desired, the passthrough area of one or more layers may be selected by the surgeon based on the specifics of the operation. If the surgeon knows that the largest stones in the patient will be 10 millimeters in diameter, the surgeon may select the netting layers which are most reliable capture all stones down to the small diameter stones which are desirably capture by the netting assembly.

In an alternate embodiment, the netting assembly may be prepackaged for insertion through the sheath 40 in a generally spiraling manner, i.e., both of the frame and the netting assembly may be twisted into a small diameter elongate configuration with the frame and the netting assembly spiraling along a generally central axis of the prepackaged assembly. This allows the frame and the netting assembly to be controllable released from the sheath 40 in manner that unfolds in a reverse spiraling manner as the netting assembly is pushing out the exit of the sheath 40. For example, the surgeon may know that insertion of the rod 90 to a selected point will result in a 50% release of the netting assembly from the sheath, and that the further insertion of another inch may result in the simultaneous rotation and extension of the netting assembly. The controlled rotation and controlled axial position of the netting assembly with respect to the sheath is to better control the configuration of the frame and the position of the netting assembly under the desired organs to serve its intended purpose. The interior of the sheath 40 may cooperate with a dog on the rod 90 to slide in within an elongate spiraling slot in the sheath to control the release of the netting assembly from the sheath 40.

In another embodiment of the present invention, the carrier sheath is provided with a plurality of through channels. One of the through channels may be sufficient to pass the netting assembly and the frame configuration control rod. A surgical tool, such as a scalpel, scissors or other cutting device, may then be passed through another of a plurality of through channels so that the surgeon may cut tissue in the area of the bile duct and gallbladder with the netting assembly already in place beneath the location of the cut to catch stones released from the gallbladder or bile duct.

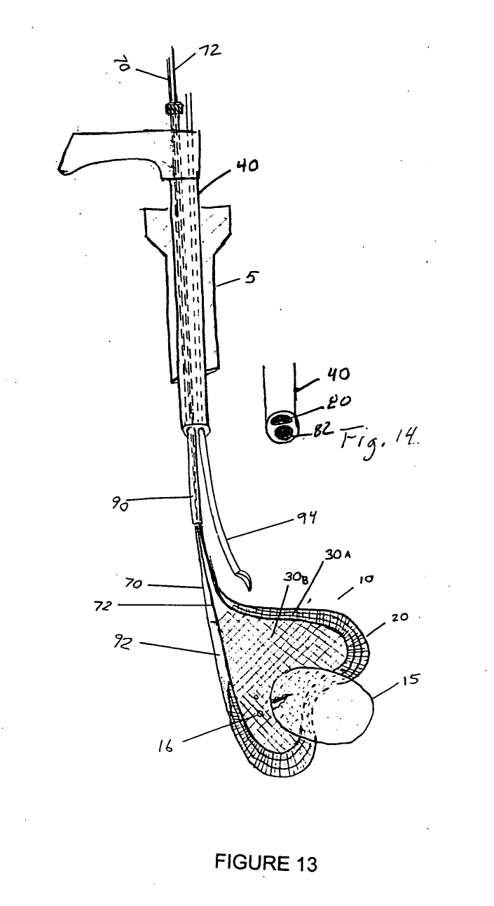

FIG. 13 is a cross-sectional view of an assembly according to the present invention, illustrating a laparoscopic port 5 which has an external end which extends axially above an external surface of the abdomen wall and an abdominal end which extends below an internal surface of the abdominal wall and into the abdominal cavity. A carrier sheath 40 includes a plurality of internal throughbores, and preferably from two to four internal throughbores, which provide conduits into the abdominal cavity. The carrier sheath 40 is provided within the laparoscopic port internal throughbore, and has a carrier sheath external end and an instrument guide abdominal end. The carrier sheath external end extends above the external surface of the abdominal wall and the carrier sheath abdominal end extends below the laparoscopic port abdominal end and proximal to the gallbladder or bile duct.

As shown in FIG. 13, the carrier sheath includes the plurality of through channels 80 and 82, with one of the channels 80 being somewhat crescent shaped for passing the netting, and the other channel having a more conventional circular cross-section, for conveying surgical tools, such as scalpel 94. FIG. 13 illustrates a control rod 90 for passing the net assembly into and out of the carrier sheath 40. At the end of control rod is a pair of wires 70, 72, which provide the frame for the netting, with separate frames provided for a lower fine netting 30A and a course top netting 30B. Each netting assembly has a generally heart-shaped configuration for more easily receiving the gallbladder 15 and one or stones. The stones 16 may thus pass through the netting 30B and be caught in the netting 30A. A fluid impermeable layer 92 may be provided in the lower layer 30A for capturing fluid released from the gallbladder, or for capturing the gallbladder and the fluid within the gallbladder. While only a portion of the layer 92 is shown, a fluid impermeable layer may be provided above or below the layer 30A, and may have the same area as netting 30A.

As shown in FIG. 13, both the outer frame wire 70 and the inner frame wire 72 pass through one of the through channels in the carrier sheath 40, and exit the top of the carrier sheath. Both the outer wire and the inner wire may be separately extendable and retractable within the through channel of the carrier sheath for changing the configuration of the frame. More particularly, the outer frame wire 72 may be retracted to be pulled at least partially over the course netting 30B supported on the inner frame wire, thereby effectively capturing the gallbladder 15 and/or stones 16 within the netting assembly.

In some applications, the sheath may be eliminated and the tools, including the netting assembly, installed through the laparoscopic port. In many applications, however, the sheath is preferred since its abdominal end may be easily positioned proximate to the gallbladder or bile duct.

While preferred embodiments of the present invention have been illustrated in detail, it is apparent that other modifications and adaptations of the preferred embodiments will occur to those skilled in the art. The embodiments shown and described are thus exemplary, and various other modifications of the preferred embodiments may be made which are within the spirit of the invention. Accordingly, it is to be expressly understood that such modifications and adaptations are within the scope of the present invention, which is defined in the following claims.

1. A laparoscopic surgical netting assembly for conducting a laparoscopic gallbladder or bile duct procedure through a laparoscopic port having an external end extending axially above an external surface of the abdominal wall and an abdominal end extending from below an internal surface of the abdominal wall and into an abdominal cavity, the laparoscopic port including an internal through bore extending between the external end and the abdominal end to provide a conduit into the abdominal cavity, and a carrier sheath received within the laparoscopic port internal through bore and having an carrier sheath external end and an instrument guide abdominal end, the carrier sheath external end extending above the external surface of the abdominal wall and the carrier sheath abdominal end extending below the laparoscopic port abdominal end and proximal to the gallbladder or bile duct, the carrier sheath including at least one through channel for conveying and deploying the surgical netting assembly, the surgical netting assembly comprising:
a collapsible and expandable frame, the frame when expanded defining a perimeter substantially greater than the frame when collapsed and passed through the at least one through channel in the carrier sheath;
a fluid permeable netting suspended on the frame for collecting stones released from the gallbladder or bile duct;
a frame control rod, the control rod being connected to the frame and extendable and retractable within the at least one through channel in the carrier sheath; and
a deployment rod for acting on a portion of the frame removed from a connection of the control rod to the frame, the deployment rod being movable within the at least one through channel in the carrier sheath which receives the control rod or another of the at least one through channel in the carrier sheath for altering a configuration of the frame when expanded.

2. A netting assembly as defined in claim 1, further comprising:
a connector for removably interconnecting the frame control rod and the frame of the netting assembly.

3. A netting assembly as defined in claim 1, further comprising:
the frame supporting a fluid impermeable layer for collecting fluid from the gallbladder or bile duct.

4. A netting assembly as defined in claim 1, wherein the expanded frame has substantially an oval configuration, when passed out of the carrier sheath, along axis of the oval con-figuration is substantially parallel to a central axis of an inward end of the carrier sheath, and the short axis of the oval configuration is substantially perpendicular to the long axis, and the long axis is from about 3″ to about 5″ in length, and the short axis is from about 1″ to about 3″ in width.

5. A netting assembly as defined in claim 4, wherein an uppermost layer of the netting is at least about ½″ below the short axis near its midpoint.

6. A netting assembly as defined in claim 1, wherein the netting comprises two or more netting layers spaced apart when the frame is in its deployed position.

7. A netting assembly as defined in claim 6, wherein a lower netting layer has a smaller passthrough area than an upper netting layer.

8. A netting assembly as defined in claim 1, wherein the expanded frame has substantially a heart-shaped configuration.

9. A netting assembly as defined in claim 1, wherein the netting comprises three or more layers, each layer spaced from an adjacent layer when the frame is in its deployed configuration.

10. A netting assembly as defined in claim 1, wherein the netting comprises strands, and the strands include loops extending in the direction substantially perpendicular to a plane of the netting.

11. A netting assembly as defined in claim 7, wherein the netting comprises a thin sponge layer.

12. A netting assembly as defined in claim 1, further comprising:
the frame having outer frame member and inner frame member spaced within the outer frame member.

13. A netting assembly as defined in claim 12, wherein the outer frame member includes an elongate outer wire, with both ends of the outer wire passing through the at least one through channel in the carrier sheath, such that the elongate outer wire may be extended and retracted within the at least one through channel in the carrier sheath;
the inner frame member includes an inner frame wire, both ends of the inner frame wire passing through the at least one through channel in the carrier sheath, such that the inner wire may be extended and retracted within the at least one through channel in the carrier sheath; and
an outer frame wire and an inner frame wire being separately extendable and retractable within the at least one through channel for changing the configuration of the frame.

14. A netting assembly as defined in claim 13, wherein the outer frame member supports a fine mesh netting, and the inner frame member supports a course mesh netting positioned above the fine mesh netting.

15. A netting assembly as defined in claim 1, further comprising:
an elongate tether secured to the frame for assisting in the retrieval of the netting assembly.

16. A laparoscopic surgical netting assembly for conducting a laparoscopic gallbladder or bile duct procedure through a laparoscopic port having an external end extending axially above an external surface of the abdominal wall and an abdominal end extending from below an internal surface of the abdominal wall and into an abdominal cavity, the laparoscopic port including an internal through bore extending between the external end and the abdominal end to provide a conduit into the abdominal cavity, the surgical netting assembly comprising:
a frame control rod extendable and retractable within the internal through bore in the laparoscopic port;
a collapsible and expandable frame, the frame when expanded defining a perimeter substantially greater than the collapsed frame; and
a fluid permeable netting suspended on the frame for collecting the gallbladder and one or more stones released from the gallbladder or bile duct.

17. A netting assembly as defined in claim 16, wherein the expanded frame has substantially an oval configuration, the long axis of the oval configuration is substantially parallel to a central axis of an inward end of the carrier sheath, and the short axis of the oval configuration is substantially perpendicular to the long axis, and the long axis is from about 3″ to about 5″ in length, and the short axis is from about 1″ to about 3″ in width.

18. A netting assembly as defined in claim 16, wherein the netting comprises two or more netting layers spaced apart when the frame is in its deployed position.

19. A netting assembly as defined in claim 16, further comprising:
a carrier sheath received within the laparoscopic port through bore and having an external end extending above the external end of the laparoscopic port and the carrier sheath abdominal end extending below the abdominal end of the laparoscopic port, at least one through channel in the carrier sheath extending from the introducer external end to the introducer abdominal end for conveying and deploying the netting assembly.

20. A netting assembly as defined in claim 16, further comprising:
the frame supporting a fluid impermeable layer for collecting fluid from the gallbladder or bile duct.

21. A netting assembly as defined in claim 16, further comprising:
a frame having outer frame member and inner frame member spaced within the outer frame member.

22. A laparoscopic surgical netting assembly for conducting a laparoscopic gallbladder or bile duct procedure through a laparoscopic port having an external end extending axially above an external surface of the abdominal wall and an abdominal end extending from below an internal surface of the abdominal wall and into an abdominal cavity, the laparoscopic port including an internal through bore extending between the external end and the abdominal end to provide a conduit into the abdominal cavity, and a carrier sheath received within the laparoscopic port internal throughbore and having an carrier sheath external end and an instrument guide abdominal end, the carrier sheath external end extending above the external surface of the abdominal wall and the carrier sheath abdominal end extending below the laparoscopic port abdominal end and proximal to the gallbladder or bile duct, the carrier sheath including a plurality of through channels, the surgical netting assembly comprising:
a collapsible and expandable frame, the frame when expanded defining a perimeter substantially greater than the collapsed frame;
a frame control rod attached to the frame and extendable and retractable within one of the plurality of through channels;
a fluid permeable netting suspended on the frame for collecting stones released from the gallbladder or bile duct; and
a surgical tool passing through another of the plurality of through channels in the carrier sheath, the surgical tool comprising one of a scalpel, a scissors and a cutting device.

23. A netting assembly as defined in claim 22, wherein the expanded frame has substantially an oval configuration, the long axis of the oval configuration is substantially parallel to a central axis of an inward end of the carrier sheath, and the short axis of the oval configuration is substantially perpendicular to the long axis, and the long axis is from about 3″ to about 5″ in length, and the short axis is from about 1″ to about 3″ in width.

24. A netting assembly as defined in claim 22, wherein the netting comprises two or more netting layers spaced apart when the frame is in its deployed position.

25. A netting assembly as defined in claim 22, further comprising: a frame having outer frame member and inner frame member spaced within the outer frame member.

26. A netting assembly as defined in claim 25, wherein the outer frame member includes an elongate outer wire, with both ends of the outer wire passing through the at least one through channel in the carrier sheath, such that the elongate outer wire may be extended and retracted within the at least one through channel in the carrier sheath;
the inner frame member includes an inner frame wire, both ends of the inner frame wire passing through the at least one through channel in the carrier sheath, such that the inner wire may be extended and retracted within the at least one through channel in the carrier sheath; and
an outer frame wire and an inner frame wire being separately extendable and retractable within the at least one through channel for changing the configuration of the frame.

27. A netting assembly as defined in claim 26, wherein the outer frame member supports a fine mesh netting, and the inner frame member supports a course mesh netting positioned above the fine mesh netting.

28. A method of recovering stones released during a laparoscopic gallbladder or bile duct procedure conducted through a laparoscopic port having an external end extending axially above an external surface of the abdominal wall and an abdominal end extending from below an internal surface of the abdominal wall and into an abdominal cavity, the laparoscopic port including an internal through bore extending between the external end and the abdominal end to provide a conduit into the abdominal cavity, and an carrier sheath received within the laparoscopic port internal through bore and having an carrier sheath external end and an carrier sheath abdominal end, the carrier sheath external end extending above the external surface of the abdominal wall and the carrier sheath abdominal end extending below the laparoscopic port abdominal end and into the gallbladder or bile duct, the carrier sheath including at least one through channel, the method comprising:
providing a collapsible and expandable frame, the frame when expanded defining a perimeter substantially greater than the collapsed frame;
suspending a fluid permeable netting on the frame for collecting stones released from the gallbladder or bile duct, while allowing fluids to passthrough the netting during retrieval of the netting assembly;
attaching the frame to a frame configuration control rod extendable and retractable through the at least one through channel; and
collecting the gallbladder and one or more stones released from the gallbladder or bile duct in the netting on the frame.

29. A method as defined in claim 28, wherein the netting comprises two or more netting layers spaced apart when the frame is in its deployed position.

30. A method as defined in claim 28, further comprising:
the frame supporting a fluid impermeable layer for collecting fluid from the gallbladder or bile duct.

31. A method as defined in claim 28, further comprising:
the frame having outer frame member and inner frame member spaced within the outer frame member.

32. A method as defined in claim 31, wherein the outer frame member includes an elongate outer wire, with both ends of the outer wire passing through the at least one through channel in the carrier sheath, such that the elongate outer wire may be extended and retracted within the at least one through channel in the carrier sheath;
the inner frame member includes an inner frame wire, both ends of the inner frame wire passing through the at least one through channel in the carrier sheath, such that the inner wire may be extended and retracted within the at least one through channel in the carrier sheath; and
an outer frame wire and an inner frame wire being separately extendable and retractable within the at least one through channel for changing the configuration of the frame.
